(19) United States
(12) Patent Application Publication
(10) Pub. No.: US 2012/0283776 A1
(43) Pub. Date: Nov. 8, 2012

(54) METHODS AND INSTRUMENTS FOR USE IN VERTEBRAL TREATMENT

(75) Inventor: Tanmay Mishra, Mountain View, CA (US)

(73) Assignee: KYPHON SARL, Neuchatel (SE)

(21) Appl. No.: 13/100,408

(22) Filed: May 4, 2011

(51) Int. Cl. A61B 17/70

(52) U.S. Cl. 606/247

(57) ABSTRACT

A spinal implant extendable across a facet joint to aid in fixation of the facet joint includes an elongate connecting member, a bone allograft, and a locking member. The elongate connecting member is sized to extend across a facet joint and includes a distal bone anchor. The bone allograft is sized for placement in a bore formed through the facet joint and configured to be placed about the elongate connecting member. The locking member includes a longitudinal bore sized to receive the elongate connecting member, and the locking member has an unlocked condition permitting movement relative the elongate connecting member and a locked condition rigidly fixing the locking member in place on the elongate connecting member. The locking member is configured to cooperate with the distal bone anchor to compress the facet joint, and the locking member is configured to lock the spinal implant across the facet joint.

### FIELD OF THE INVENTION

The present invention relates generally to the field of fixation mechanisms for facet joint stabilization.

### BACKGROUND

The vertebrae in a patient's spinal column are linked to one another by the intervertebral disc and the facet joints. Each vertebra has four facet joint surfaces: a pair of articulating surfaces located on the left side, and a pair of articulating surfaces located on the right side. Each facet joint is a synovial joint consisting of two overlapping articulating surfaces, an superior articular process of one vertebra and an inferior articular process of the vertebra directly above it. The biomechanical function of each facet joint is to guide and limit the motion of the spinal motion segment. These functions can be disrupted by disc or bone degeneration, dislocation, fracture, injury, trauma-induced instability, osteoarthritis, and surgery. Such damage to the facet joint can result in pain, a misaligned spine, impinged nerves, and loss of mobility. In certain cases, partial or complete immobilization of one or more facet joints by intervertebral stabilization is desirable to alleviate the patient's symptoms.

Intervertebral stabilization is designed to prevent or restrict relative motion between the vertebrae of the spine. One method of intervertebral stabilization is to directly fasten one or both of the facet joints in a spinal motion segment together, thereby limiting intervertebral motion. From a surgical perspective, the facet joint is more easily accessible than the vertebral body or the pedicles, thus reducing operative time, decreasing blood loss, decreasing incision size, reducing incidence of reoperation, and decreasing the risk of potential deleterious effects on nearby anatomic structures, including the spinal cord.

In order to provide effective fixation of the facet joint, a fixation device should create compression between the two articular processes. The compression, which causes or enhances immobilization of the joint by encouraging stability through the joint, should be maintained over a significant length of time. In addition, the device must work to prevent loosening of the device. Because the facet joint is designed to be a mobile, weight-bearing joint, forces will continue to be transmitted through the joint after the implantation of a fixation device. Without a specific way to prevent loosening of the device, loosening will likely occur as a result of the micromotion caused by such forces. Once the device has loosened, the device may begin to protrude or regress from the bone, causing pain, joint damage, or danger to the surrounding tissues.

Surgeons have used various fixation devices, including bone screw assemblies, to immobilize the facet joint. Examples of facet fixation devices currently used to stabilize the spine include trans-lamina facet screws and trans-facet pedicle screws. The previously proposed facet fixation devices, however, have presented significant shortcomings. Both trans-lamina facet screws and trans-facet pedicle screws can be difficult to surgically place, have long trajectories, and may deleteriously interfere with the local anatomy once implanted. In addition, though a standard fully threaded bone screw may be sufficient for adjoining two bone surfaces, a fully threaded screw may not be capable of creating a desirable amount of compression between two bone surfaces. Any compression generated between the bone surfaces would be limited to the compressive forces generated by the screw threads themselves. Further, a bone screw may loosen overtime. When a screw is over-tightened and threads are stripped within the bone, or when threads strip over time as a result of micromotion, the compressive force between the facet joint surfaces will diminish and loosening will likely occur. To prevent loosening, still other bone screws are designed such that a portion of the screw expands within the bone after the device is implanted. However, the expansion of the device within bone generates great stress on the bone, making this device ill-suited for use in the relatively small bones of the facet joint. In an attempt to simultaneously maintain compression and prevent loosening, nut-and-bolt type assemblies have been presented as a method of facet joint immobilization. In this type of assembly, a threaded bolt or screw is passed through the facet joint and a nut with mating threads is placed around the distal end of the bolt or screw. Though this approach is successful in maintaining compression and preventing loosening, this approach mandates a surgical procedure that is more invasive than desired because the nut must be introduced to the back side of the facet joint.

Thus, though various systems in the prior art have attempted to achieve effective facet joint fixation, none of the prior art systems enable facet joint fixation through a minimally invasive, compressive, and stable facet fixation device. Accordingly, there is a need for instrumentation and techniques that facilitate the safe and effective stabilization of facet joints. Therefore, it would be advantageous to provide a system and method of facet joint fixation that can be implanted simply, accurately, and quickly, while providing suitable stabilization to the facet joint.

The device and methods disclosed herein overcome one or more of the shortcomings discussed above and/or in the prior art.

### SUMMARY

The present invention relates to devices and methods for accomplishing bone fixation, and more particularly in some embodiments, to devices and methods for fixation of spinal facet joints.

In one exemplary aspect, the present disclosure is directed to a spinal implant extendable across a facet joint to aid in fixation of the facet joint. The implant may comprise an elongate connecting member sized to extend across a facet joint, a bone allograft, and a locking member. The elongate connecting member may have a distal end comprising a distal bone anchor. The bone allograft may be sized for placement in a bore formed through the facet joint and configured to be placed about the elongate connecting member. The locking member may include a longitudinal bore sized to receive the elongate connecting member, and may have an unlocked condition permitting movement relative the elongate connecting member and a locked condition rigidly fixing the locking member in place on the elongate connecting member. The locking member may be configured to cooperate with the distal bone anchor to compress the facet joint, and the locking member may be configured to lock the spinal implant across the facet joint.

In another exemplary aspect, the present disclosure is directed to a spinal implant for fixation of a facet joint. The implant may comprise an elongate connecting member sized to extend across a facet joint, a bone allograft, a locking member, and a stabilization member. The elongate connecting member may have a distal end comprising a distal bone anchor. The bone allograft may be sized for placement in a bore formed through the facet joint and configured to be placed about the elongate connecting member. The locking member may include a longitudinal bore sized to receive the elongate connecting member, and may have an unlocked condition permitting movement relative the elongate connector and a locked condition rigidly fixing the locking member in place on the elongate connecting member. The locking member may be configured to cooperate with the distal bone anchor to compress the facet joint, and the locking member may be configured to lock the spinal implant across the facet joint. The stabilization member may include a bone contacting surface and an opposing surface, and the stabilization member may be configured to seat the locking member on the opposing surface. The stabilization member may include a hole extending therethrough sized to receive the elongate connecting member, wherein the stabilization member slides over a portion of the elongate connecting member such that the portion extends through the hole.

In another exemplary aspect, the present disclosure is directed to a method for fixation of a facet joint, the facet joint having a superior articular process and an inferior articular process. The method may comprise: forming a drill hole through the facet joint, inserting an elongate connecting member having a distal anchor into the facet joint and advancing the elongate connecting member and the distal anchor into the facet joint until the elongate connecting member spans the facet joint, drilling a well circumferentially around the elongate connector member, packing the well with a bone allograft, sliding a locking member over the elongate connector member such that locking member contacts the inferior articular process, and compressing the locking member around the elongate connector member to stabilize the facet joint.

Further aspects, forms, embodiments, objects, features, benefits, and advantages of the present invention shall become apparent from the detailed drawings and descriptions provided herein.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a lateral view of a portion of the lumbar spine with a portion of a facet joint in cross-section, showing a spinal implant disposed within the facet joint in accordance with a first embodiment of the present disclosure.

FIG. 2 is a perspective view of the spinal implant shown in FIG. 1.

FIG. 3 is a highly simplified drawing of a portion of a vertebral arch, showing a delivery cannula positioned through the skin and against the inferior articular process of a facet joint.

FIG. 4 is a lateral, partial cross-sectional view of a spinal motion segment showing an elongate connecting member and a distal anchor of the spinal implant of FIG. 1 inserted into a facet joint.

FIG. 5 is a lateral, partial cross-sectional view of a spinal motion segment showing a cannulated drill positioned around the elongate connecting member and against the inferior articular process.

FIG. 6 is a lateral, partial cross-sectional view of a spinal motion segment showing a drilled intrafacet cavity.

FIG. 7 is a lateral, partial cross-sectional view of a spinal motion segment showing a bone allograft within the intrafacet cavity.

FIG. 8 is a lateral, partial cross-sectional view of a spinal motion segment showing insertion of a locking member around the elongate connecting member.

FIG. 9 is a lateral view of a spinal motion segment showing a spinal implant inserted into and fixed against the facet joint.

FIG. 10 is a perspective view of a spinal implant in accordance with a second embodiment of the present disclosure.

FIG. 11a is a highly simplified, partial cross-sectional view of the first embodiment of the spinal implant in its final, expanded state.

FIG. 11b is a highly simplified, partial cross-sectional view of the second embodiment of the spinal implant in its final, expanded state.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

This disclosure describes implants and methods for stabilizing a facet joint. The implants described herein are structurally designed to span the facet joint and, due to the placement of a bone allograft across the joint, create stable fixation through fusion. The implants fasten one or both of the facet joints in a spinal motion segment together, thereby limiting intervertebral motion and alleviating the patient's symptoms.

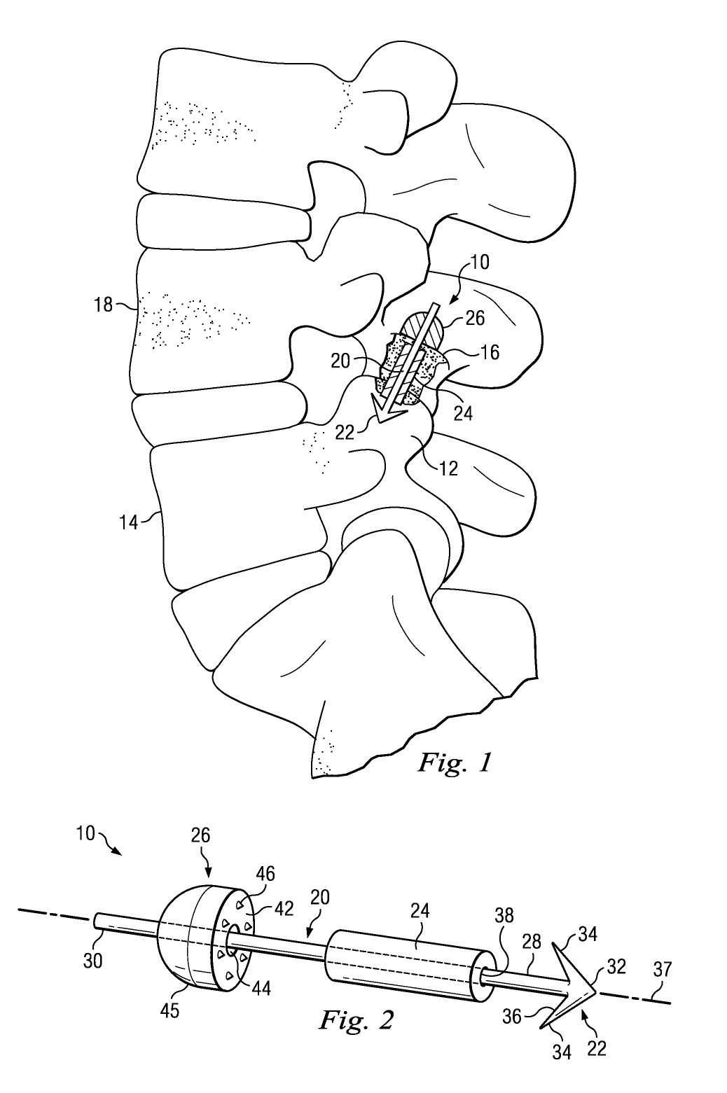

FIG. 1 illustrates an implant 10 according to an exemplary embodiment of the present invention for fixing, stabilizing, and/or immobilizing a joint. The spinal implant 10 is shown implanted within a facet joint formed by a superior articular process 12 of one vertebra 14 and an inferior articular process 16 of the vertebra 18 immediately above. The implant 10 can also be utilized to stabilize other joints besides the facet joint. The implant 10 includes an elongate connecting member 20, a distal anchor 22, a bone allograft 24, and a locking member 26. FIG. 1 shows the implant 10 inserted into the facet joint such that the distal anchor 22 protrudes outside and lateral to the superior articular process 12 while the elongate connecting member 20 and the bone allograft 24 remain within the bony tissue of the facet joint. Accordingly, the elongate connecting member 20 is disposed through both the superior articular process 12 and the inferior articular process 16 through a drill hole formed through both the processes 12, 16. The bone allograft 24 promotes bone fusion between the superior articular process 12 and the inferior articular process 16. The locking member 26, which is sized to have a wider diameter than the drill hole, is positioned flush against the exterior surface of the inferior articular process 16. The implant 10 provides stabilization and immobilization of the facet joint formed by the processes 12, 16 through compressive forces applied by the distal anchor 22 and the locking member 26. In addition, the amount of compressive force applied by the implant 10 can vary with the position of the locked locking member 26 relative to the distal anchor 22. The closer to the distal anchor 22 that the locking member 26 is locked, the greater the compressive forces exerted on the facet joint.

FIG. 2 illustrates the exemplary implant 10 in an expanded state. As indicated above, the implant 10 includes the elongate connecting member 20, the distal anchor 22, the bone allograft 24, and the locking member 26. In the embodiment shown in FIG. 2, the elongate connecting member 20 is approximately cylindrical and configured to be received within the prepared drill hole through the superior articular process 12 and the inferior articular process 16 of the facet joint. The elongate connecting member 20 is made of a flexible and durable biocompatible material configured as a cable. For example, the elongate connecting member 20 can be constructed of surgical stainless steel, titanium, cobalt-chromium alloy, Nitinol, ultra-high molecular weight polyethylene, poly(tetraflouroethylene) or poly(tetraflouroethene) (PTFE), polyethylene terephthalate (PET), or any other biocompatible material as is known in the art of medical device manufacture. Alternatively, the elongate connecting member could be configured as a wire, a braid, or a rod. Optionally, the elongate connecting member 20 can be constructed of a radiolucent material, such as polyaryletheretherketone (PEEK) or the like, such that it can be medically imaged and visualized. Further, the elongate connecting member 20 can be treated with growth factors, stem cells, or any other device coating known in the art, to be selected based on the desired outcome of the procedure. In some embodiments, the elongate connecting member 20 is substantially taut and rigid, while in other embodiments, the elongate connecting member 20 can flex. In these embodiments, the elongate connecting member 20 is flexible enough to be positioned within a prepared drill hole formed through the facet joint, but is rigid enough to immobilize the superior articular process 12 and the inferior articular process 16 with respect to one another. In some examples, the elongate connecting member 20 is configured to flex or bend laterally, but is configured to substantially resist axial elongation.

As shown in FIG. 2, the elongate connecting member 20 extends from a proximal portion 30 to a distal portion 28 which is attached to the distal anchor 22. In some embodiments, the distal portion 28 may extend to integrally form the distal anchor 22. The elongate connecting member 20 is of a length suitable to fit through the facet joint from the exterior surface of the inferior articular process 16 to the exterior surface of the superior articular process 12. The elongate connecting member 20 can include a variety of lengths and dimensions as required for different spinal morphologies.

The distal anchor 22 may be configured to have an unexpanded configuration or state and an expanded configuration or state. In the unexpanded configuration or state, the distal anchor 22 may be sized and configured to pass through a pilot hole formed through the facet joint. In the expanded configuration or state, the distal anchor 22 may be sized and configured as a hook-like structure to anchor the implant 10 and resist axial regression through the pilot hole. In the embodiment pictured in FIG. 2, the distal anchor 22 in an expanded state has an arrowhead-like configuration including an exterior surface 32, at least two flanges 34, and bone-engaging surfaces 36. In the example shown in FIG. 2, the flanges 34 are moveable between two positions: an insertion position or unexpanded state wherein the flanges 34 are approximately parallel with a longitudinal axis 37 of the elongate connecting member 20, and a bone-engaging position or expanded state wherein the flanges 34 are angled with respect to the longitudinal axis 37 of the elongate connecting member 20. More specifically, in the insertion, unexpanded state, the flanges 34 are positioned generally flush against the distal portion 28 of the connecting member 20. Upon emerging from the prepared drill hole through the facet joint, the flanges 34 flare away from the connecting member 20 and the distal anchor 22 assumes a bone-engaging, expanded state. At least a portion of the bone-engaging surfaces 36 of the distal anchor 22 then engages the exterior surface of the superior articular process 12. The material composition of the distal anchor 22 resiliently biases the flanges 34 toward the bone-engaging, expanded state. In this example, the distal anchor 22 is made of a flexible, surgical-grade material that is configured to allow extensive short-term deformation without permanent deformation, cracks, tears, or other breakage. In particular, in this example, the distal anchor is made of a shape memory alloy having a memory shape in the expanded configuration. In other embodiments, the distal anchor 22 is formed of an elastic material allowing the flanges 34 to elastically deform to an unexpanded state to fit through the drilled hole, and spring back to an expanded state when the distal anchor 22 advances clear of the hole. In the embodiment pictured in FIG. 2, the exterior surface 32 is smooth. However, in other embodiments, the exterior surface 32 can include features that engage bony tissue. The features can resemble screw threads or any other configuration that would interface with and provide friction with bony tissue. The features can include structures of various sizes, dimensions, shapes, and configurations.

As the embodiment pictured in FIG. 2 shows, the implant 10 also includes a bone allograft 24. The bone allograft 24 has a generally cylindrical shape having generally planar and circular ends and a generally cylindrical sidewall. The bone allograft 24 includes a centrally disposed and cylindrically shaped bore 38. The diameter of bore 38 is slightly larger than the diameter of the elongate connecting member 20, such that the elongate connecting member 20 is slidable within the bore 38. In some embodiments, the bone allograft 24 is composed of a bone dowel. In other embodiments, the bone allograft 24 is composed of loose allograft material, such that the allograft material surrounds the elongate connecting member 20 when the implant 10 is in final position across the facet joint.

As the embodiment pictured in FIG. 2 shows, the implant 10 also includes a locking member 26. The locking member 26 is approximately cylindrical, and has a proximal surface 40, a bone-engaging surface 42, and a centrally disposed and cylindrically shaped longitudinal bore 44. The proximal surface 40 can be flat or rounded or have a variety of configurations compatible with the adjacent anatomical tissue. In the example shown, the proximal surface 40 is rounded to avoid edges that may introduce additional tissue trauma. The bone-engaging surface 42 can be flat or curved or have a variety of configurations compatible with the exterior surface of the inferior articular process 16. In some embodiments, the locking member 26 can be approximately spherical. In some embodiments, the locking member 26 can be non-continuous in that a longitudinal slot comprising the length of the locking member 26 extends from the sidewall 45 to the bore 44.

The implant 10 pictured in FIG. 2 has features 46 that extend perpendicularly from the bone-engaging surface 42. Here, the features 46 are triangular protrusions capable of stabilizing the locking member 26 against the exterior surface of the inferior articular process 16. Pressure can be exerted on the locking member 26 to embed the features 46 in the surface of the inferior articular process 16. The locking member 26 may include any number of features 46. The features 46 can include structures of various sizes, dimensions, shapes, and configurations. Further, a single locking member 26 can include features 46 of different sizes, dimensions, shapes, and configurations. In addition, the locking member 26 can include any orientation of features 46 on the bone-engaging surface 42. For example, the features can be equally spaced around the circumference of the bone-engaging surface, thereby allowing the locking member 26 to engage the inferior articular process 16 and adding to the overall stability of the implant 10. In other embodiments, the features 46 can extend at acute or obtuse angles from the bone-engaging surface 42.

The bore 44 extends longitudinally through the locking member 26 from the proximal surface 40 to the bone-engaging surface 42. The diameter of bore 38 is slightly larger than the diameter of the elongate connecting member 20, such that the elongate connecting member 20 is slidable within the bore 44. The inner surface of the bore 44 can be textured such that the bore 44 of locking member 26 grips the connecting member 20.

The locking member 26 is formed of a deformable and durable surgical-grade material. For example, the locking member 26 can be constructed of surgical stainless steel, titanium, cobalt-chromium alloy, Nitinol, ultra-high molecular weight polyethylene, poly(tetraflouroethylene) or poly(tetraflouroethene) (PTFE), polyethylene terephthalate (PET), or any other deformable biocompatible material as is known in the art of medical device manufacture. Optionally, the locking member 26 can be constructed of a radiolucent material, such as polyaryletheretherketone (PEEK) or the like, such that it can be medically imaged and visualized. In one example, after the implant 10 is positioned across the facet joint, the locking member 26 is fixedly secured to the elongate connecting member 20 by crimping the locking member 26 to the connecting member 20 such that the desired amount of compression is achieved across the facet joint.

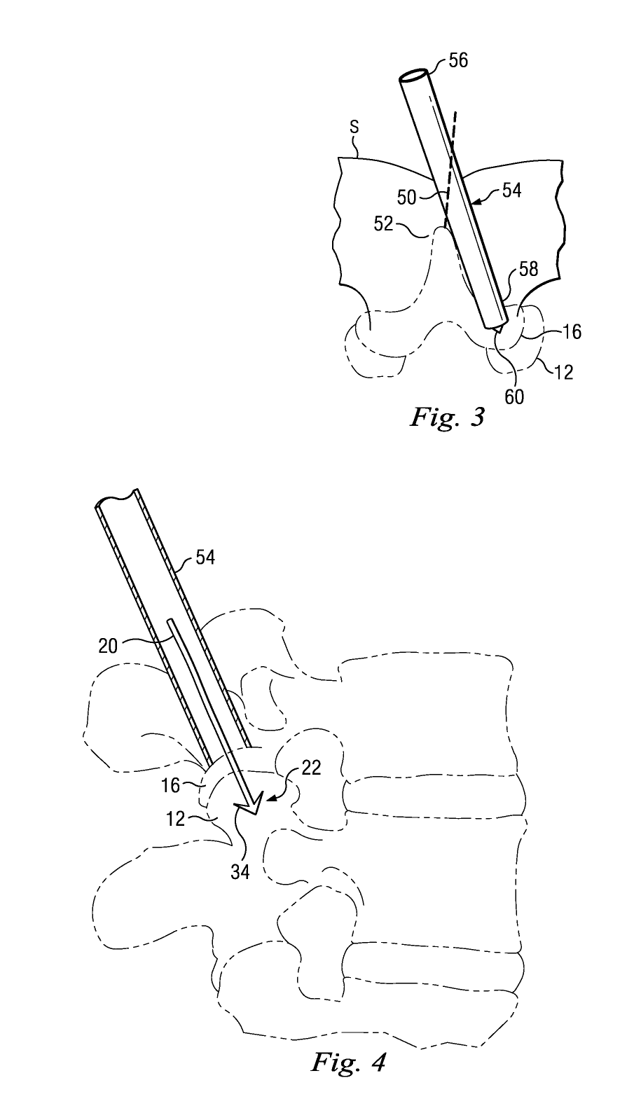

The implant 10 is utilized to stabilize and/or immobilize the facet joint by limiting the motion between the superior articular process 12 and the inferior articular process 16. The implant 10 is assembled and implanted in the following manner, described with reference to FIGS. 3-9. In FIGS. 3-9, the vertebrae are depicted in dashed lines to indicate that the drawings illustrate the two-dimensional positional relationship of the implant 10 relative to the three-dimensional vertebral structures.

First, access to the facet joint is gained through any suitable surgical technique using any suitable device. Advantageously, referring to FIG. 3, the implant 10 can be implanted through a minimally invasive surgical procedure involving a single midline incision 50 through the skin S over the spinous process 52 of the vertebra 18 superior to the target facet joint. In FIG. 3, a custom delivery cannula 54 (that is part of a delivery device) is shown resting against the inferior articular process 16 after being inserted through a midline incision 50. The cannula 54 is operable to route the elongate connecting member 20, the distal anchor 22, the bone allograft 24, and the locking member 26 into correct positions relative to the facet joint. The cannula 54 is made of a surgical-grade material, and in particular stainless steel, though other materials are suitable. The cannula 54 is cylindrical with an longitudinal passage extending along its entire length from a proximal end 56 to a distal end 58. The diameter of the cannula 54 is larger than the diameter of the implant 10 in an expanded configuration such that the implant 10 in an expanded configuration is slidable within the passage of the cannula 54. Further, the diameter of the cannula 54 is such that the bone allograft 24 and the locking member 26 are slidable within the passage of the cannula 54.

The distal end 58 of the delivery cannula 54 includes at least one docking feature 60 that extends in the same plane as the longitudinal passage of the cannula 54. The docking feature 60 is capable of stabilizing the cannula 54 to an anatomical structure. For example, the docking feature 60 can serve as a docking point on which the cannula 54 may securely rest against or penetrate the inferior articular process 16, thereby preventing the cannula 54 from slipping from the surface of the inferior articular process 16 during the implantation procedure. Pressure can be exerted on the delivery cannula 54 to temporarily embed the feature in the surface of the inferior articular process 16. The delivery cannula 54 may include any number of such docking features 60. The docking features 60 can include structures of various sizes, dimensions, shapes, and configurations. Further, a single cannula 54 can include docking features 60 of different sizes, dimensions, shapes, and configurations. In addition, the cannula 54 can include any orientation of such docking features 60 on the bone contacting surface. For example, the docking features 60 can be equally spaced around the circumference of the distal end of the cannula 54. In some embodiments, the docking feature 60 may be angled away from the side of the cannula 54.

A hole is then formed through the facet joint by any of various mechanisms as are known in the art. An exemplary mechanism for forming a hole through the facet joint involves directing a drill through the cannula 54, positioning the drill against the exterior surface of the inferior articular process 16, and drilling a continuous hole through both the inferior articular process 16 and the superior articular process 12. The hole is formed in a desired location to provide optimal stabilization/immobilization of the facet joint. The hole is dimensioned to allow passage of the elongate connecting member 20 and the distal anchor 22. Other mechanisms for forming the hole are also contemplated. For example, an 11-gauge needle could be used to “punch” a hole through the facet joint. After the hole is prepared, the drill or other instrument used to form the hole is withdrawn from the cannula 54.

Referring to FIG. 4, the elongate connecting member 20 and the distal anchor 22 are passed through the cannula 54 to be inserted into the prepared drill hole in an insertion, unexpanded state. In this example, when in the insertion, unexpanded state, the flanges 34 of the distal anchor 22 are positioned generally flush against the distal portion 28 of the connecting member 20 such that the connecting member 20 and the folded distal anchor 22 possess a smaller diameter than the diameter of the prepared drill hole. The connecting member 20 is then pushed forward through the prepared drill hole until the distal anchor 22 emerges through the superior articular process 12. Upon emerging from the prepared drill hole, the flanges 34 of the distal anchor 22 flare away from the connecting member 20 and the distal anchor 22 assumes a bone-engaging, expanded state. More specifically, once the distal anchor 22 has passed all the way through the inferior articular process 16, across any gap between the inferior articular process and the superior articular process, and all the way through the superior articular process 12, the flanges 34 of the distal anchor 22 flare out from their insertion, unexpanded position—in substantial alignment with the longitudinal axis 37 of the elongate connecting member 20—into their bone-engaging, expanded position at an angle with the longitudinal axis 37 of the elongate connecting member 20, as shown in FIG. 2. At least a portion of the bone-engaging surfaces 36 of the distal anchor 22 then engages the exterior surface of the superior articular process 12. Once in position, the elongate connecting member 20 and the distal anchor 22 function to properly align and hold the superior articular process 12 against the inferior articular process 16. It is worth noting that although the elongate connecting member 20 is shown with a limited length, in some embodiments, the elongate connecting member 20 has an overall length greater than the length of the cannula 54. Accordingly, the elongate connecting member 20 may extend out the proximal end of the cannula 54 for easy access by the surgeon.

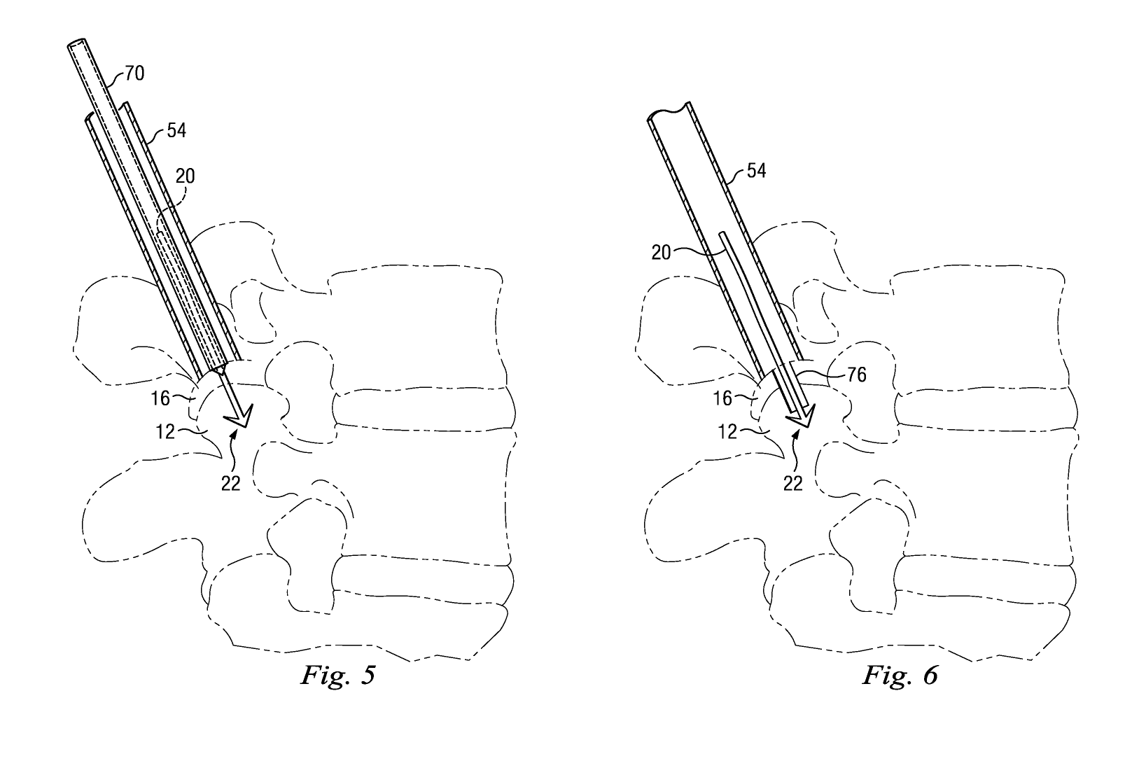

Referring to FIG. 5, a cannulated drill 70 is advanced through the cannula 54 and around the proximal portion 30 of the elongate connecting member 20 until it rests against the surface of the inferior articular process 16. Using the cannulated drill, an intrafacet cavity 76 is drilled around the elongate connecting member 20 all the way through the inferior articular process 16, and partially into the superior articular process 12 such that the intrafacet cavity 76 extends to a depth in the range of, for example, one third to one half of the thickness of the superior articular process 12, as shown in FIG. 6. In some embodiments, the cannulated drill 70 is configured to debride the cartilaginous tissue between the inferior articular process 16 and the superior articular process 12, thereby “bloodying” the intrafacet cavity 76 and creating a favorable environment for the bone allograft 24. In some embodiments, the cannulated drill 70 includes a suction channel, which is operatively arranged to remove excess bone and tissue debris created by the drilling process. After the intrafacet cavity 76 is prepared, the cannulated drill 70 is withdrawn from the cannula 54.

Referring to FIG. 6, the intrafacet cavity 76 is shown extending all the way through the inferior articular process 16 and extending partially into the superior articular process 12. The intrafacet cavity 76 is shaped and sized to accommodate the bone allograft 24.

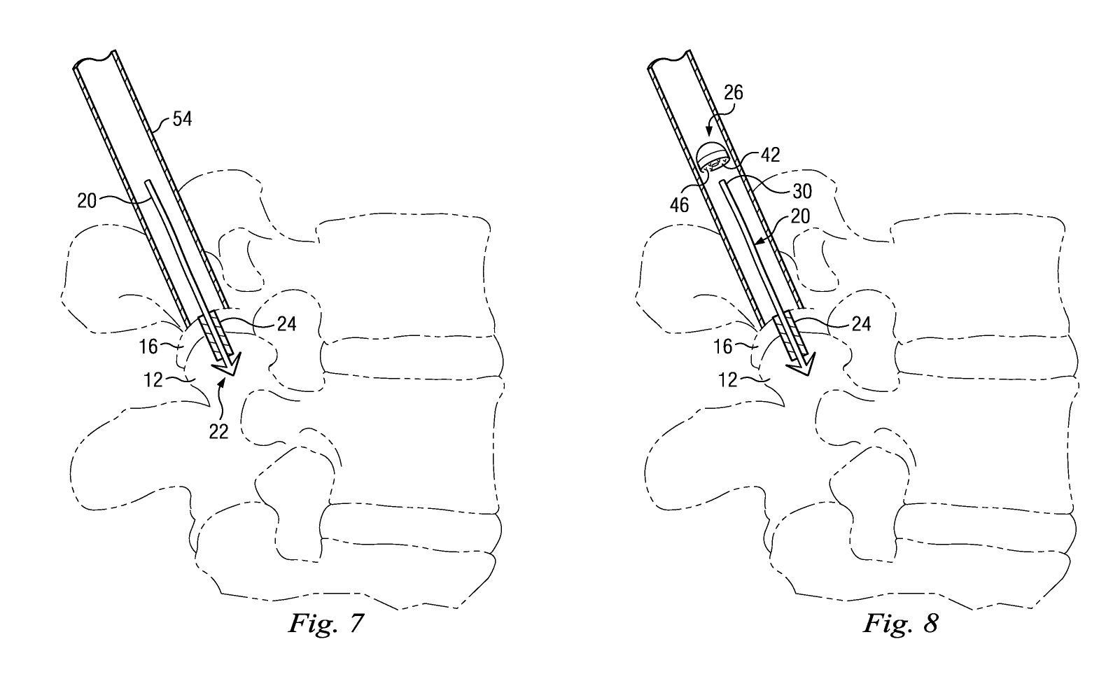

Referring to FIG. 7, the bone allograft 24 is passed through the cannula 54 and slid over the proximal portion 30 of the elongate connecting member 20. In particular, the bore 38 of the bone allograft 24 is positioned to encircle the proximal portion 30 of the elongate connecting member 20, and then the bone allograft 24 is slid down the elongate connecting member to rest in the intrafacet cavity 76. The bone allograft 24 promotes bone fusion between the superior articular process 12 and the inferior articular process 16.

Referring to FIG. 8, after insertion of the bone allograft 24 into the intrafacet cavity 76, the locking member 26 is passed through the cannula 54 and slid over the proximal portion 30 of the elongate connecting member 20. In particular, the bore 44 of the locking member 26 is positioned to encircle the proximal portion 30 of the elongate connecting member 20, and then the locking member 26 is slid down the elongate connecting member to rest against the exterior surface of the inferior articular process 16. The features 46 on the bone-engaging surface 42 of the locking member 26 engage with the exterior surface of the inferior articular process 16 such that the locking member is stabilized against the exterior surface of the inferior articular process 16. Pressure can be exerted on the locking member 26 to embed the features 46 in the surface of the inferior articular process 16. Due to the diameter of the locking member 26, which is greater than the diameter of the intrafacet cavity 76 into which the bone allograft 24 is inserted, the locking member 26 provides a mechanism by which the bone allograft is isolated within the facet joint and the implant 10 is fixed within the facet joint, thereby stabilizing the facet joint. Tension is provided by pulling the proximal portion 30 of the elongate connecting member 20 through the locking member 26. As tension is provided, the locking member 26 is pushed against the inferior articular process 16 to achieve the desired alignment and compression of the facet joint. Specifically, the proximal portion 30 of the elongate connecting member 20 is pulled in the longitudinal axis 37 in a direction away from the distal anchor 22 while the locking member 26 is simultaneously pushed against the inferior articular process such that the desired alignment and compression of the superior articular process 12 against the inferior articular process 16 is achieved. After the desired alignment and amount of compression are realized, the locking member 26 is fixedly secured to the elongate connecting member 20 by crimping the locking member 26 to the connecting member 20 such that the desired alignment and amount of compression is maintained across the facet joint.

The implant 10 provides stabilization and immobilization of the facet joint formed by the processes 12, 16 through compressive forces applied by the distal anchor 22 and the locking member 26. In addition, the amount of compressive force applied by the implant 10 can vary with the position of the locked locking member 26 relative to the distal anchor 22. The closer to the distal anchor 22 that the locking member 26 is locked, the greater the compressive forces exerted on the facet joint.

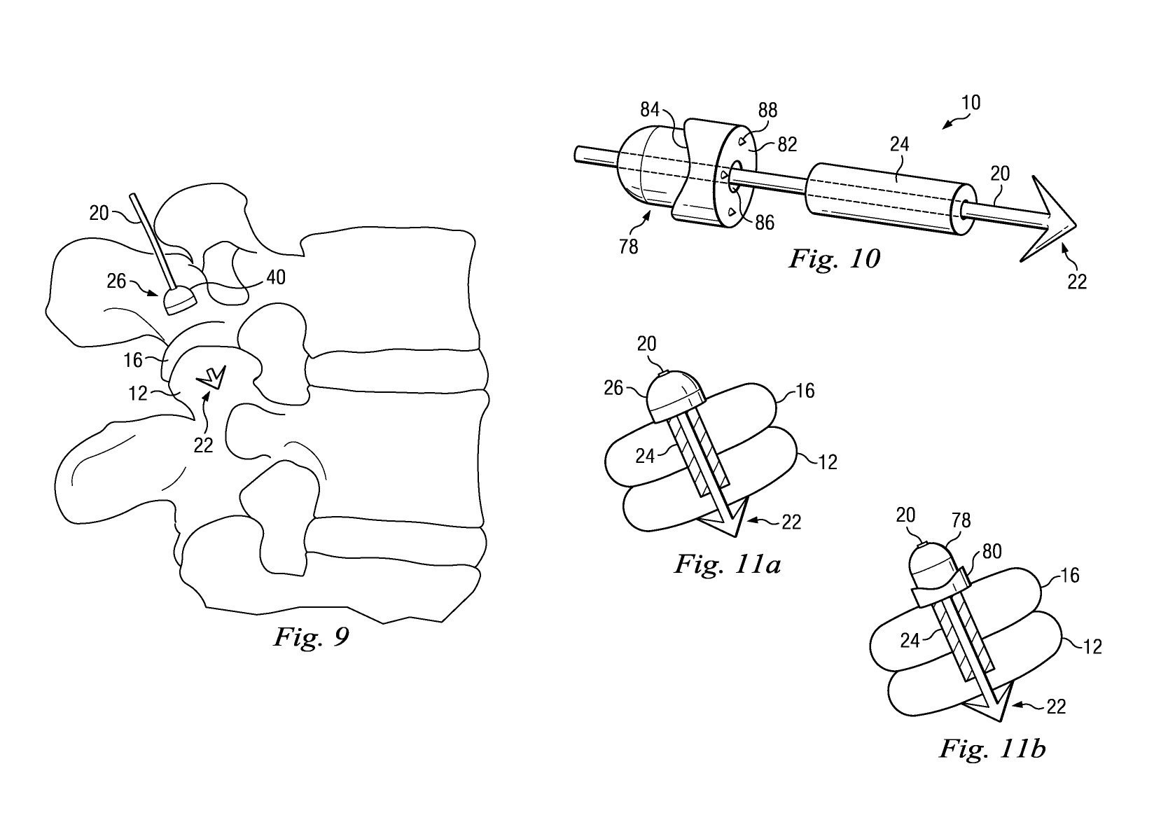

FIG. 9 shows the spinal implant 10 inserted into and fixed against the facet joint. The proximal portion 30 of the elongate connecting member 20 is shown extending proximally through the locking member 26. The surgeon clips the proximal portion 30 of the elongate connecting member 20 such that the elongate connecting member 20 no longer substantially extends past the proximal surface 40 of the locking member 26, as shown in FIG. 11a. FIG. 11a shows the spinal implant 10 spanning across the inferior articular process 16 and the superior articular process 12 in its final, expanded state.

FIG. 10 illustrates a second embodiment of the spinal implant 10 in its expanded state. In the embodiment illustrated in FIG. 10, the implant 10 includes the elongate connecting member 20, the distal anchor 22, the bone allograft 24, a locking member 78, and a stabilization member 80. The locking member 78 is substantially similar in shape and size to the locking member 26, but the locking member 78 includes a distal surface configured to interface with a proximal surface of the stabilization member 80. In this embodiment, the stabilization member 80 is configured as a gimbaled washer. As such, the stabilization member 80 includes a proximal surface 84 configured to seat the locking member 78. The stabilization member 80 also includes a bone contacting surface 82 being configured to engage the exterior surface of the inferior articular process 16. The stabilization member 80 includes a hole 86 extending from the bone contacting surface 82 to the proximal surface 84 sized to encircle the elongate connecting member 20. The diameter of the hole 86 is less than the diameter of the locking member 78, thereby allowing the stabilization member 80 to seat the substantially hemispherical bottom portion of the locking member 78 on the concave proximal surface 84. As such, the hemispherical bottom portion of the locking member 78 being seated in the stabilization member 80 enables polyaxial motion of the locking member 78 relative to the stabilization member 80. Providing such a polyaxial coupling allows greater versatility of the implant 10 because the stabilization member 80 and the locking member 78 can adjust to anatomical structures of various shapes, thereby allowing for a more personalized and precise fit of the implant 10.

The stabilization member 80 includes at least one feature 88 extending perpendicularly from the bone contacting surface 82 capable of stabilizing the stabilization member 80 against the inferior articular process 16. The features 88 can include structures of various sizes, dimensions, shapes, and configurations. Further, a single stabilization member 80 can include features 88 of different sizes, dimensions, shapes, and configurations. For example, the features 88 can be configured as any one of spikes, teeth, serrations, grooves, or ridges. Referring to FIG. 10, the stabilization member 80 includes a plurality of features 88 configured as triangular protrusions adapted to pierce the outer portion of the inferior articular process 16. The stabilization member 80 may include any number of such features. In addition, the stabilization member 80 can include any orientation of such features 88 on the bone contacting surface 82. For example, the features 88 can be equally spaced around the circumference of the bone-contacting surface 82, thereby allowing the stabilization member 80 to engage the inferior articular process 16 and adding to the overall stability of the implant 10. In other embodiments, the features 88 can extend at acute or obtuse angles from the bone contacting surface 82.

FIG. 11b shows the second embodiment of the spinal implant 10, as illustrated in FIG. 10, spanning across the inferior articular process 16 and the superior articular process 12 in its final, expanded state.

The devices, systems, and methods described herein provide an improved and more accurate system of facet joint stabilization. Applicants note that the procedures disclosed herein are merely exemplary and that the systems and methods disclosed herein may be utilized for numerous other medical processes and procedures. Although several selected embodiments have been illustrated and described in detail, it will be understood that they are exemplary, and that a variety of substitutions and alterations are possible without departing from the spirit and scope of the present invention, as defined by the following claims.

We claim:

1. A spinal implant extendable across a facet joint to aid in fixation of the facet joint, the spinal implant comprising:
an elongate connecting member sized to extend across a facet joint, the elongate connecting member having a distal end comprising a distal bone anchor;
a bone allograft sized for placement in a bore formed through the facet joint and configured to be placed about the elongate connecting member; and
a locking member including a longitudinal bore sized to receive the elongate connecting member, the locking member having an unlocked condition permitting movement relative the elongate connecting member and a locked condition rigidly fixing the locking member in place on the elongate connecting member, the locking member being configured to cooperate with the distal bone anchor to compress the facet joint, and the locking member being configured to lock the spinal implant across the facet joint.

2. The spinal implant of claim 1 wherein the distal bone anchor has an expanded configuration and an unexpanded configuration, the expanded configuration being sized to prevent regression of the distal bone anchor through the facet joint.

3. The spinal implant of claim 1 wherein the elongate connecting member is one of a rigid rod, a wire, and a cable, the elongate connector substantially resisting all axial extension.

4. The spinal implant of claim 1 wherein the distal bone anchor comprises threads shaped and configured to engage bone and prevent axial removal from a bone structure.

5. The spinal implant of claim 1 wherein the distal bone anchor comprises a hook-like structure configured to engage bone and prevent axial removal from a bone structure.

6. The spinal implant of claim 1 wherein the distal bone anchor is capable of self-expansion into a predetermined shape.

7. The spinal implant of claim 6 wherein the predetermined shape is an arrowhead configuration.

8. The spinal implant of claim 6 wherein the distal bone anchor is composed of a material having shape memory.

9. A spinal implant for fixation of a facet joint, the spinal implant comprising:
an elongate connecting member sized to extend across a facet joint, the elongate connecting member having a distal end comprising a distal bone anchor;
a bone allograft sized for placement in a bore formed through the facet joint and configured to be placed about the elongate connecting member; and
a locking member including a longitudinal bore sized to receive the elongate connecting member, the locking member having an unlocked condition permitting movement relative the elongate connector and a locked condition rigidly fixing the locking member in place on the elongate connecting member, the locking member being configured to cooperate with the distal bone anchor to compress the facet joint, the locking member being configured to lock the spinal implant across the facet joint; and
a stabilization member including a bone contacting surface and an opposing surface, the stabilization member being configured to seat the locking member on the opposing surface, the stabilization member including a hole extending therethrough sized to receive the elongate connecting member, wherein the stabilization member slides over a portion of the elongate connecting member such that the portion extends through the hole.

10. The spinal implant of claim 9 wherein the distal bone anchor is capable of self-expansion into a predetermined shape.

11. The spinal implant of claim 10 wherein the predetermined shape is an arrowhead configuration.

12. The spinal implant of claim 10 wherein the distal bone anchor is composed of a material having shape memory.

13. The spinal implant of claim 9 wherein the longitudinal bore of the locking member includes a textured inner surface configured to grip the elongate connecting member.

14. The spinal implant of claim 9 wherein the stabilization member is configured to seat the locking member such that the locking member is capable of polyaxial movement relative to the stabilization member.

15. The spinal implant of claim 9 wherein the bone contacting surface of the stabilization member includes a plurality of protrusions configured to engage bone.

16. The spinal implant of claim 15 wherein the plurality of protrusions includes at least one of spikes, teeth, serrations, grooves, or ridges.

17. A method for fixation of a facet joint, the facet joint having a superior articular process and an inferior articular process, the method comprising:
forming a drill hole through the facet joint, inserting an elongate connecting member having a distal anchor into the facet joint and advancing the elongate connecting member and the distal anchor into the facet joint until the elongate connecting member spans the facet joint;
drilling a well circumferentially around the elongate connector member;
packing the well with a bone allograft;
sliding a locking member over the elongate connector member such that locking member contacts the inferior articular process; and
compressing the locking member around the elongate connector member to stabilize the facet joint.

18. The method of claim 17 further including the steps of:
making a midline incision above a superior spinous process to access the facet joint and providing a delivery cannula having a proximal end and a distal end, the distal end including a protrusion such that the protrusion holds the cannula against the inferior articular process prior to making a drill hole through the facet joint;
sliding a stabilization member over the elongate connector member such that the stabilization member grips the inferior articular process after the step of packing the well with a bone allograft; and
sliding a locking member over the elongate connector member such that locking member contacts the stabilization member prior to compressing the locking member around the elongate connector member to stabilize the facet joint.

19. The method of claim 17 wherein the step of inserting comprises advancing the elongate connecting member and the distal anchor so that the distal anchor emerges from the drill hole and expands to an expanded configuration.

20. The method of claim 17 further including the step of compressing the facet joint between the locking member and the distal anchor prior to the step of compressing the locking member around the elongate connector member to stabilize the facet joint.
